SEQUENCE LISTING (2 sequences)

SEQ ID NO: 1 - protein, 9 aa, Mus musculus
SFVMDYYET

SEQ ID NO: 2 - protein, 13 aa, Mus musculus
AVVFLTKRGRQIC

(19) United States
(12) Patent Application Publication
(10) Pub. No.: US 2020/0172609 A1
(43) Pub. Date: Jun. 4, 2020

(54) METHOD FOR PREVENTING OR TREATING ATHEROSCLEROSIS

(71) Applicants: Chang-Fen HUANG, Atlanta, GA (US); NATIONAL YANG-MING UNIVERSITY, Taipei City (TW); TAIPEI VETERANS GENERAL HOSPITAL, Taipei City (TW); VIVASOLIS BIOTECHNOLOGY CO., LTD., Taipei City (TW)

(72) Inventor: Jaw-Wen CHEN, Taipei City (TW)

(73) Assignees: NATIONAL YANG-MING UNIVERSITY, Taipei City (TW); TAIPEI VETERANS GENERAL HOSPITAL, Taipei City (TW); VIVASOLIS BIOTECHNOLOGY CO., LTD., Taipei City (TW); Chang-Fen HUANG, Atlanta, GA (US)

(21) Appl. No.: 16/637,383

(22) PCT Filed: Aug. 10, 2018
(86) PCT No.: PCT/US2018/046328

§ 371 Date: Feb. 7, 2020

(30) Foreign Application Priority Data
Aug. 10, 2017 (TW) 106127097
Aug. 25, 2017 (CN) PCT/CN2017/098977

(51) Int. Cl. C07K 16/24; A61P 9/10; G01N 33/68

(52) U.S. Cl. CPC C07K 16/24; A61P 9/10; A61K 2039/505; C07K 2317/76; G01N 33/6863

(57) ABSTRACT

The preset invention relates to preventing, arresting, reversing or treating atherosclerosis, comprising a step of: administering to a subject in need thereof a therapeutically effective amount of a macrophage inflammatory protein-1 beta (MIP-1β) inhibitor.

### FIELD OF THE INVENTION

The present invention relates to a new method for preventing or treating atherosclerosis, in particular a method for preventing or treating atherosclerosis using an anti-MIP-1β antibody.

### BACKGROUND OF THE INVENTION

Atherosclerosis is a chronic inflammatory disorder of artery leading to cardiovascular morbidity and mortality. Inflammatory cytokines and chemokines play important roles in the pathogenesis and complications of atherosclerosis. Endothelial dysfunction caused by various risk factors including hyperglycemia, hypertension, low density lipoprotein (LDL) and others are regarded as the key mechanism for atherogenesis. Then, circulating LDL could enter the sub-endothelial layer where it may be oxidized to oxidized LDL (ox-LDL) as one of the key components of atheroma. On the other hand, upon stimuli, endothelial cells, together with other vascular cells, may produce various inflammatory mediators, including adhesion molecules and cytokines, such as tumor necrosis factor (TNF)-α, interleukin (IL)-1, IL-6, and so on. They could promote endothelial adhesion of circulating leukocytes, direct the migration of bound leukocytes into intima, mature the monocytes to macrophages, and enhance the lipid uptake of macrophage to form the lipid core in atheroma plaques. Importantly, atheroma with a thin fibrous cap, a large necrotic core, and a high content of leucocyte are more inflammatory and vulnerable to rupture, suggesting a high-risk phenotype for acute cardiovascular events. It was suggested to identify novel anti-inflammatory strategy to stabilize atheroma plaques for the prevention of clinical events.

Although the cause of atherosclerosis is unknown, atherosclerosis may be treated with the heart-healthy lifestyle changes, medicines, and medical procedures or surgery. Normally, the goals of treatment include lowering the risk of blood clots forming, preventing atherosclerosis-related diseases, relieving symptoms and widening or by passing plaque-clogged arteries. Treatment of established disease may include medications to lower cholesterol such as statins, blood pressure medication, or medications that decrease clotting, such as aspirin. A number of procedures may also be carried out such as percutaneous coronary intervention, coronary artery bypass graft, or carotid endarterectomy.

It is still desirable to develop an effective method for treating atherosclerosis.

### SUMMARY OF THE INVENTION

It is unexpectedly found in the present invention that a macrophage inflammatory protein-1 beta (MIP-1β) inhibitor, such as a specific MIP-1β antibody, could retard the progression and promote the stabilization of atheroma plaques in a mice model of atherosclerosis. Accordingly, the present invention provides a new approach for preventing, arresting, reversing or treating atherosclerosis through the inhibition of MIP-1β.

In one aspect, the invention provides a method for preventing, arresting, reversing or treating atherosclerosis, comprising a step of administering to a subject in need thereof an therapeutically effective amount of an anti-MIP-1β inhibitor.

In one further aspect, the invention provides a method for preventing or treating a inflammatory cardiovascular disease or disorder, comprising a step of administering to a subject in need thereof a therapeutically effective amount of a MIP-1β inhibitor, wherein the cardiovascular disease or disorder is selected from the group consisting hyperlipidaemia, hypercholesterolaemia, heart attack, stroke, and coronary heart disease.

In another aspect, the invention provides a method for treating or preventing atherosclerosis, the method comprising the steps of:

(1) providing a sample of the subject and determining the level of MIP-1β in the sample, and
(2) administering to said subject, if the subject is found to have a higher level of MIP-1β in the sample than a normal level of a healthy population, a therapeutically effective amount of a MIP-1β inhibitor.

In one embodiment of the invention, the therapeutically effective amount of anti-MIP-1β inhibitor is the amount sufficient to reduce atherosclerotic lesions or plaques.

In one embodiment of the invention, the therapeutically effective amount of anti-MIP-1β inhibitor is the amount sufficient to retard the progression and promote the stabilization of atheroma plaques.

In one embodiment of the invention, the therapeutically effective amount of anti-MIP-1β inhibitor is the amount sufficient to lower blood lipids, triglyceride, cholesterol and non-high-density lipoprotein.

In one yet aspect, the invention provides a method for lowering blood lipids, triglyceride, cholesterol or non-high-density lipoprotein, comprising a step of administering to a subject in need thereof a therapeutically effective amount of a MIP-1β inhibitor.

In one further yet aspect, the invention provides a use of an anti-MIP-1β antibody for manufacturing a medicament for preventing, arresting, reversing or treating atherosclerosis.

In one further aspect, the invention provides a pharmaceutical composition for preventing, arresting, reversing or treating atherosclerosis comprising a therapeutically effect amount of anti-MIP-1β antibody, a binding protein or peptide or a fragment thereof which is capable of binding to MIP-1β, and a pharmaceutically acceptable carrier.

In one embodiment of the invention, the MIP-1β inhibitor is an anti-MIP-1β antibody, or a fragment thereof.

In one particular example of the invention, the MIP-1β inhibitor is a binding protein or peptide capable of binding to a MIP-1β or a fragment thereof, such as a peptide binding to an amino acid sequence of SFVMDYYET (SEQ ID NO: 1), or AVVFLTKRGRQIC (SEQ ID NO: 2).

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiment which is presently preferred. It should be understood, however, that the invention is not limited to this embodiment.

In the drawings:

FIG. 1: The effects of anti-MIP-1β antibody on cytokines, including the levels of MIP-1β (FIGS. 1A, 1B and 1C), IL-6 (FIG. 1D), TNF-α (FIG. 1E) in serum (n=6 in each group); wherein the Western blotting of aorta tissue and quantification of relative expression of MIP-1β in aorta were obtained respectively (#1 represents upper aorta; #2 represents lower aorta, quantification of relative expression of MIP-1β=MIP-1β expression/β-actin expression; # P<0.05, ## P<0.01 compared with the IgG₂ₐ isotype control group).

FIG. 2: The effects of anti-MIP-1β antibody on the metabolic parameters including blood glucose levels (FIG. 2A), total cholesterol levels (FIG. 2B), triglyceride levels (FIG. 2C), non-HDL levels (FIG. 2D) in serum and body weight (FIG. 2E) (n=6 in each group), wherein the Westerb blotting of LXR expression in liver and statistical analyses for the Western blotting were obtained (quantification of relative expression of LXR=LXR expression/β-actin expression (FIG. 2F; n=3). (# P<0.05, ## P<0.01 compared with the IgG₂ₐ isotype control group).

FIG. 3: Anti-MIP-1β antibody reduced the atherosclerosis lesion size, reduced the necrotic area, and increased the fibrous cap thickness; wherein the quantification of the plaque area (μm²) (FIG. 3A, n=6 in each group). Quantification of fibrous cap thickness (μm) (FIG. 3B, n=6 in each group). Quantification of necrotic area/plaque area (%) (FIG. 3C, n=6 in each group). (# P<0.05, ## P<0.0/compared with the IgG₂ₐ isotype control group).

FIG. 4: Anti-MIP-1β antibody reduced the number of macrophage and MIP-1β expressions in plaques; wherein Quantification of average F4/80 signal/DAPI (FIG. 4A, n=6 in each group). Quantification of average MIP-1β signal/DAPI (FIG. 4B, n=6 in each group) (# P<0.05, ## P<0.01 compared with the IgG₂ₐ isotype control group).

FIG. 5: Anti-MIP-1β antibody reduced MMP2 and MMP9 expressions in plaques; wherein Quantification of MMP2 positive area/plaque area (%) (FIG. 5A, n=6 in each group). Quantification of MMP9 positive area/plaque area (%) (FIG. 5B, n=6 in each group) (# P<0.05, ## P<0.01 compared with the IgG_{2A} isotype control group).

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

As used herein, the term “macrophage inflammatory protein-1 beta” or “MIP-1β,” also known as chemokine (C—C motif) ligand 4 (CCL4) refers to one of the ligands of chemokine (C—C motif) receptor 5 (CCR5), id major factor produced by macrophages after they are stimulated with bacterial endotoxin, and crucial for immune responses towards infection and inflammation, and can induce the synthesis and release of other pro-inflammatory cytokines such as interleukin 1 (IL-1), IL-6 and TNF-α from fibroblasts and macrophages.

As used herein, the term “MIP-1β inhibitor” refers to an agent or a molecule that decreases/regulates the level of MIP-1β; and/or directly or indirectly decreases or inhibits the activity of MIP-1β. Examples of the MIP-1β-inhibitor include (1) a MIP-1β modulating agent/compound that decreases the level of MIP-1β, or homologs thereof; (2) a MIP-1β agent/compound that suppresses the expression of MIP-1β, such as a siRNA, an antisense nucleic acid, or a ribozyme targeted to the MIP-1β; (3) an agent that inhibits transcription of MIP-1β; and (3) an agent that modulates the transcription of genes encoding MIP-1β, such as an agent destabilizing the mRNAs. In an exemplary embodiment, a MIP-1β-inhibitor may be a compound that decreases at least one biological activity of MIP-1β by at least about 10%, 25%, 50%, 75%, 100%, or more.

In a particular embodiment of the invention, the MIP-1β-inhibitor is a molecule which is capable of binding to MIP-1β. For instance, according to some embodiments of the present invention, an agent/molecule capable of inhibiting the activity of MIP-1β, such as an anti-MIP-1β antibody.

As used herein, the term “antibody” means an immunoglobulin molecule or a fragment of an immunoglobulin molecule having the ability to specifically bind to a particular antigen. The term “antibody” herein is used in the broadest sense and specifically includes a full-length monoclonal antibody, a polyclonal antibody, a multispecific antibody (e.g., a bispecific antibody), and antibody fragments thereof, as long as they exhibit the desired biological activity.

As used herein, the term “antibody fragment” refers to a portion of a full-length antibody, preferably antigen-binding or variable regions thereof. Examples of antibody fragments include Fab, Fab′, F(ab)₂, F(ab′)₂, F(ab)₃, Fv (typically the VL and VH domains of a single arm of an antibody), single-chain Fv (scFv), dsFv, Fd fragments (typically the VH and CH1 domain), and dAb (typically a VH domain) fragments; VH, VL, and VhH domains; minibodies, diabodies, triabodies, tetrabodies, and kappa bodies; camel IgG; and multispecific antibody fragments formed from antibody fragments, and one or more isolated CDRs or a functional paratope, where isolated CDRs or antigen-binding residues or polypeptides can be associated or linked together so as to form a functional antibody fragment.

In the present invention, it was confirmed that a macrophage inflammatory protein-1 beta (MIP-1β) inhibitor, such as a specific MIP-1β antibody, could retard the progression and promote the stabilization of atheroma plaques in a mice model of atherosclerosis.

Accordingly, the invention provides a method for preventing, arresting, reversing, or treating atherosclerosis, comprising a step of administering to a subject in need thereof a therapeutically effective amount of an anti-MIP-1β inhibitor.

Further, the invention provides a method for preventing or treating an inflammatory cardiovascular disease or disorder, comprising a step of administering to a subject in need thereof a therapeutically effective amount of a MIP-1β inhibitor.

In addition, the invention provides a method for treating or preventing atherosclerosis, the method comprising the steps of:

(1) providing a sample of the subject and determining the level of MIP-1β in the sample, and
(2) administering to said subject, if the subject is found to have a higher level of MIP-1β in the sample than a normal level of a healthy population, a therapeutically effective amount of a MIP-1β inhibitor.

On the other hand, the invention provides a method for lowering blood lipids, triglyceride, cholesterol or non-high-density lipoprotein, comprising a step of administering to a subject in need thereof a therapeutically effective amount of a MIP-1β inhibitor.

In one embodiment of the invention, the MIP-1β inhibitor is an anti-MIP-1β antibody, or a fragment thereof. In certain embodiments, the MIP-1β-antibody is a monoclonal antibody specifically binding to MIP-1β, called as “anti-MIP-1 antibody.” In one embodiment, the anti-MIP-1β monoclonal antibody has the binding specificity for a functional fragment of MIP-1β.

In one particular example of the present invention, the MIP-1β-inhibitor is a monoclonal antibody that binds to the antigen determinant fragment of MIP-1β, which is a peptide having an amino acid sequence of SFVMDYYET (SEQ ID NO:1), the 46th to 54th amino acid residues of MIP-1β; or a peptide having an amino acid sequence of AVVFLTKRGRQIC (SEQ ID NO:2), the 62nd to 74th amino acid residue of MIP-1β.

In one particular embodiment of the present invention, the MIP-1β-inhibitor is a monoclonal antibody or a functional fragment thereof; preferably a humanized antibody or a human antibody.

Pharmaceutical compositions for use in accordance with the present invention may be formulated in a conventional manner using one or more physiologically acceptable carriers comprising excipients and auxiliaries which facilitate processing of the active compounds into preparations which can be used pharmaceutically. Proper formulations, including (but not limited to) oral compositions such as tablets, capsules, powders and the like, parenteral compositions such as aqueous solutions for subcutaneous, intramuscular or intraperitoneal injection, and lyophilized powders combined with a physiological buffer solution just before administration, are formulated depending upon the chosen route of administration.

According to the present invention, the method may comprise further administering a second therapeutically active agent, such as proteins, peptides, polysaccharides, lipids, nucleic acid molecule, synthetic organic molecules, hormones, antibiotics, antivirals, antifungals, vasoactive compounds, immunomodulatory compounds, vaccines, local anesthetics, antiangiogenic agents, and antibodies.

The term “atherosclerosis” is given its ordinary meaning in the art and refers to a disease of the arterial wall in which the layer thickens, causing narrowing of the channel and thus, impairing blood flow. Atherosclerosis may occur in any area of the body, but can be most damaging to a subject when it occurs in the heart, brain or blood vessels leading to the brain stem. Atherosclerosis includes thickening and hardening of artery walls or the accumulation of fat, cholesterol and other substances that form atheromas or plaques.

As used herein, the term “inflammatory cardiovascular disease or disorder” refers to a cardiovascular disease or disorder caused by inflammation. In the present invention, an inflammatory cardiovascular disease or disorder is selected from the group consisting of hyperlipidaemia, hypercholesterolaemia, heart attack, stroke, coronary heart disease, and a cardiovascular disorder

As used herein, a “subject” refers to any mammal (e.g., a human), such as a mammal that comprises at least one tissue lumen or hollow organ. Examples include a human, a non-human primate, a cow, a horse, a pig, a sheep, a goat, a dog, a cat, or a rodent such as a mouse, a rat, a hamster, or a guinea pig. Generally, or course, the invention is directed toward use with humans. A subject may be a subject diagnosed with the disease or condition or otherwise known to have the disease or condition (e.g., atherosclerosis). In some embodiments, a subject may be diagnosed as, or known to be, at risk of developing a disease or condition.

The “therapeutically effective amount” as used herein means that amount of a compound, material, or composition comprising a compound of the present invention which is effective for producing some desired therapeutic effect in a subject at a reasonable benefit/risk ratio applicable to any medical treatment. Accordingly, a therapeutically effective amount prevents, minimizes, or reverses disease progression associated with a disease or condition. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

The present invention will now be described more specifically with reference to the following examples, which are provided for the purpose of demonstration rather than limitation.

### EXAMPLES

Materials and Methods

Animal Model

Apolipoprotein E-deficient (ApoE KO) mice are well validated model of atherosclerosis that follows a pattern of progression similar to that of human disease. Wild-type (WT) and ApoE KO mice on a C57BL/6 background were purchased from the Jackson Laboratories (ME, U.S.A.). The mice were fed with standard chow diet or Western diet. The water was given ad libitum. The mice were maintained on a 12-h light and dark cycle.

From 5 weeks of age, male control C57BL/6 mice were fed a standard chow and male ApoE KO mice were fed a Western diet (20% fat, 0.15% cholesterol; AIN-76A) for a given period of time (5-16 weeks). After 12 weeks on standard chow or the Western diet, mice were sacrificed. Additionally, ApoE KO mice fed a Western diet were treated with a mouse anti-MIP-1β monoclonal antibody (#46907) [MAB451](1 or 10 μg per mouse, i.p. R&D Systems) or IgG2a isotype control [MAB006] 3 times per week up to 4 weeks. The animal study project was approved by the Institutional Animal Care and Use Committee of School of National Yang-Ming University, Taipei, Taiwan. All experiments conformed to the relevant regulatory standards.

Tissue Harvesting

Mice were anesthetized and left ventricle was perfused with PBS (10 ml) with an exit through the severed right femoral artery. The heart and aorta (section between the heart and the bifurcation of an iliac artery) were harvested, cleaned of adventitial fat and fixed in 4% paraformaldehyde solution overnight. The heart and aorta were embedded into Paraffin.

Histologic Staining

After the heart and aorta were embedded into Paraffin, for the quantitation of atherosclerosis, 8 μm serial sections of aortic sinus or arch were stained with hematoxylin and eosin to determine lesion size. Elastica van Gieson staining was used for the visualization of vascular elastic fibers and collagen. Furthermore, it was used to determine necrotic core area as well as fibrous cap thickness. Quantification analysis of plaques areas was assessed with Motic Images Plus 2.0 software. The necrotic core area and fibrous cap thickness were quantitated by Image J software.

Immunohistochemical Staining

Immunohistochemical assays were performed with the following primary antibodies: rat F4/80 antibody (Cl-A3-1) [NB600-404] (1:50 dilutions; Novus), rabbit MMP-9 antibody [PAS-13199] (1:50 dilution; Thermo scientific), rabbit MMP-2 antibody [PA1-16667] (4 μg/ml dilution; Thermo scientific), goat MIP-1β antibody (M20) [sc-1387] (1:50 dilution; Santa Cruz Biotechnologies). Secondary antibodies used in these assays were purchased from Jackson ImmunoResearch Laboratories, Inc. The reaction was visualized by staining with 3, 3-diaminobenzidine (DAB) or fluorescence (FITC). Quantification analysis of fluorescent was assessed with Metamorph software [Immunohistochemical analysis was quantitated by Image J software].

Biochemical Indexes

Blood samples from mice were harvested at time points of 12, 14, 16 weeks old mice after a 5-hour fast. Placed blood samples at room for 2 hours. Then blood samples were centrifuged for 25 minutes at 2100 rpm, and sera were transferred and stored at −80° C. Levels of total cholesterol (TC), triglycerides (TGs), and non-high-density lipoprotein (non-HDL) were determined by using Automated Clinical Chemistry Analyzer (FUJI DRI-CHEM 4000i); blood glucose was measured by Optium Xceed.

Enzyme Linked Immunosorbant Assay

Levels of IL-6, TNF-α, and MIP-1β in serum were measured by R&D systems ELISA kits. The assay employs the quantitative sandwich enzyme immunoassay technique. An affinity purified polyclonal antibody specific for mouse IL-6, TNF-α or MIP-1β had been pre-coated onto a microplate individually. Standard, control, and sample were pipetted into wells and any mouse IL-6, TNF-α or MIP-1β present were bound by the immobilized antibody. After washing away any unbound substances, an enzyme-linked polyclonal antibody specific for mouse IL-6, TNF-α or MIP-1β was added to the wells. Following a wash to remove any unbound antibody-enzyme reagent, a substrate solution was added to the wells. The enzyme reaction yielded a blue product that turned yellow when the stop solution was added. The intensity of the color measured is in proportion to the amount of mouse IL-6, TNF-α or MIP-1β bound in the initial step. The sample values were then read off the standard curve.

Western Blotting

There were three groups of blood vessels tissues. The blood vessels dissected into two sections for different groups. Different groups of blood vessel tissues were rinsed into RIPA lysis buffer (50 mM Tris-HCl pH 7.4, 1% NP-40, 0.5% Na-deoxycholate, 0.1% SDS, 150 mM NaCl, 2 mM EDTA, 50 mM NaF) containing protease inhibitor cocktail (Calbiochem) for 1-hour incubation on ice. After centrifugation, the supernatant contained whole cell lysates. Protein concentrations were measured by BCA assay (Thermo). Protein was subjected to 12% SDS-PAGE in running buffer (25 mM Tris pH 8.8, 192 mM glycine, 0.1% SDS). PVDF membrane was activated by methanol before electroblotting separated protein onto a PVDF membrane in transfer buffer (25 mM Tris pH 8.8, 192 mM glycine, 20% methanol). Membranes were probed with monoclonal antibodies directing to MIP-1β (R&D antibodies), and β-actin (Chemicon) at 4° C. overnight. After incubation with secondary antibody, the probed proteins were visualized by using chemiluminescence detection reagents according to the manufacturer's instructions.

Statistical Analyses

Data were presented as mean±standard deviation (SD). Statistical differences were assessed by one-way analysis of variance (ANOVA) and unpaired t tests in treatment groups and control group. P value less than 0.05 was regarded as significant.

### Example 1: Elevated Levels of MIP-1β in Serum and Aorta of Atherosclerotic Mice were Reduce after 4 Weeks Antibody Treatment

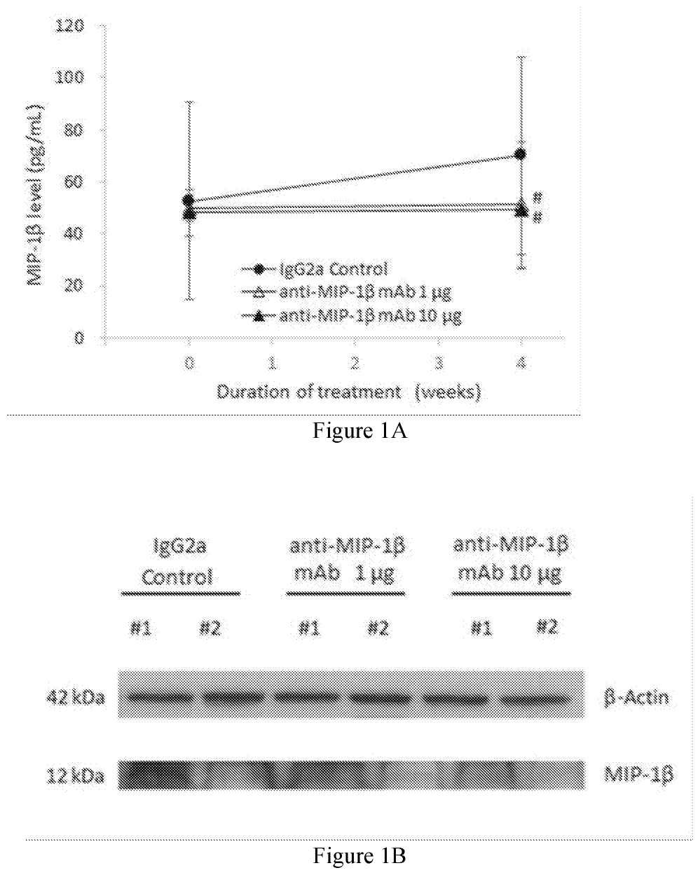

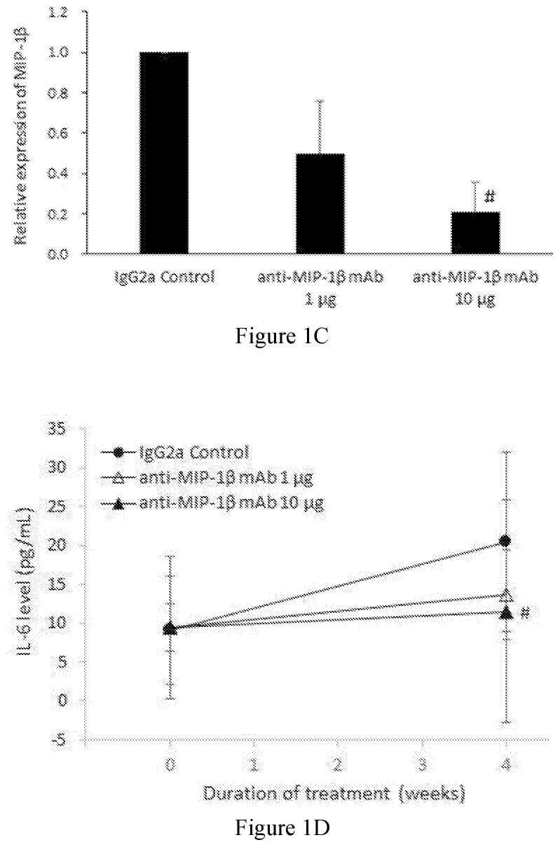

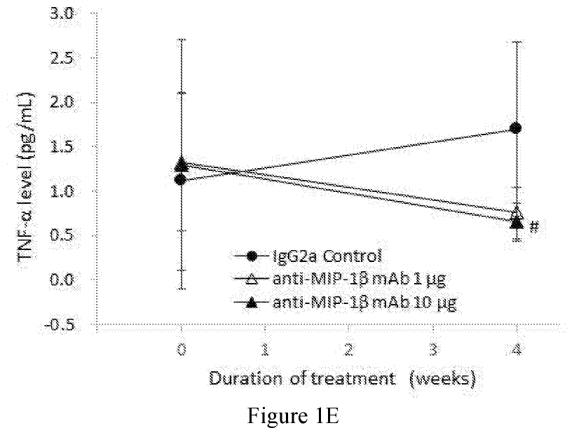

This study defines the levels of MIP-1β in the serum of animal model. The ELISA results indicate that the MIP-1β level was elevated in IgG control group (n=6) from 46.82±22.9 to 71.87±35.79 pg/mL. (FIG. 1A). On the other hand, after a 4-week treatment, the level of MIP-1β in serum in both 1 μg and 10 μg anti-MIP-1β antibody-treated groups (n=6 for each group) remain unchanged (1 μg: 47.75±10.13˜46.67±27.57 pg/mL; 10 μg: 51.17±24.20˜46.45±21.70 pg/mL) (FIG. 1A).

At the time of sacrifice, this study dissected the aorta into two sections for western blot assay. The lower sections of aorta had more MIP-1β expression than upper sections. Moreover, the MIP-1β protein level in the whole aorta significantly decreased in 10 μg antibody-treated group (˜78%) (n=6 for each group) (FIGS. 1B and C).

### Example 2. MIP-1β Neutralization Attenuated Pro-Inflammatory Factors in Circulating and Atherosclerotic Plaques

Atherosclerosis and increased risk of thromboembolic complications have been associated with increased circulating levels of IL-6 and TNF-α. To confirm the effects of anti-MIP-1β antibody on proinflammatory factors IL-6 and TNF-α level, the ELISA was performed and the results show the levels of IL-6 were reduced compared to IgG control group in anti-MIP-1β antibody-treated groups (IgG: 9.07±7.01˜20.40±10.54 pg/mL; 1 μg: 9.73±9.16˜13.68±5.75 pg/mL; 10 μg: 9.06±7.17˜11.47±14.31 pg/mL) (FIG. 1D). Compared to IgG control group, the levels of TNF-α were also reduced in anti-MIP-1β antibody-treated groups (IgG:1.13±0.93˜1.70±0.98 pg/mL; 1 μg:1.16±0.69˜0.76±0.29 pg/mL; 10 μg: 1.16±0.82˜0.59±0.40 pg/mL) (FIG. 1E).

### Example 3: MIP-1β Neutralization Effect on Metabolic Parameters

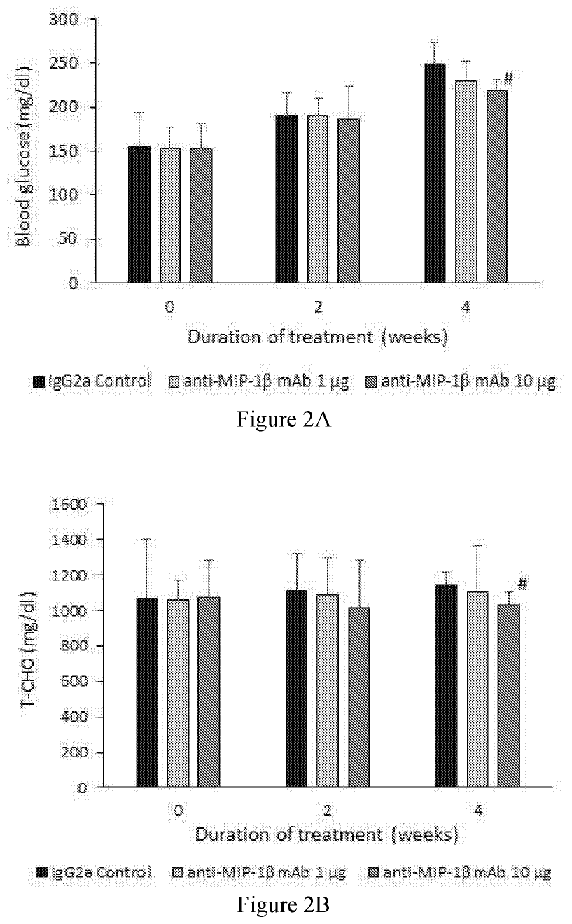

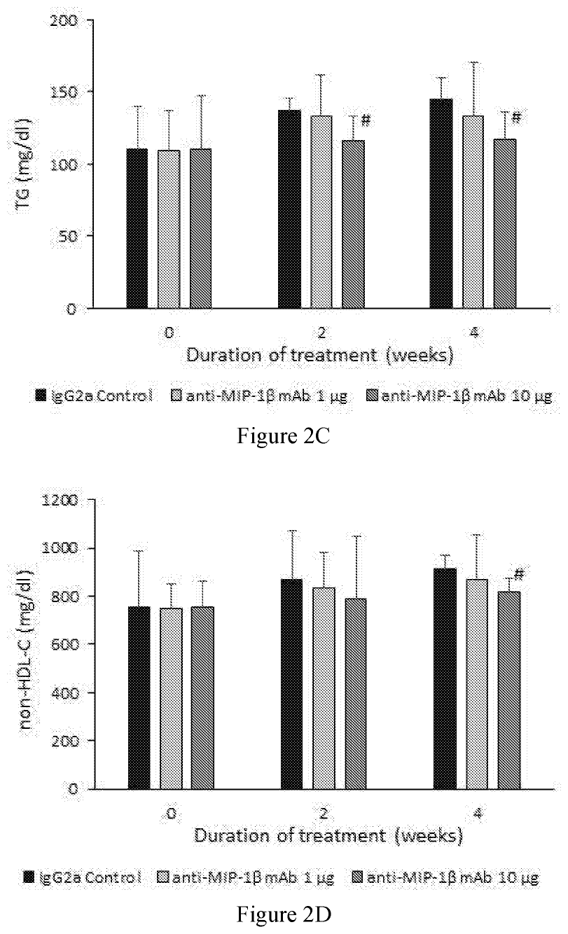

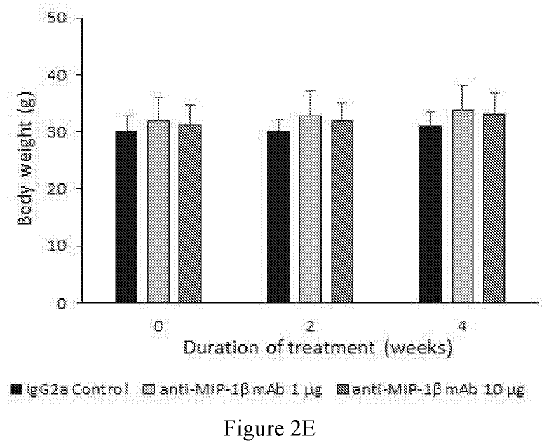

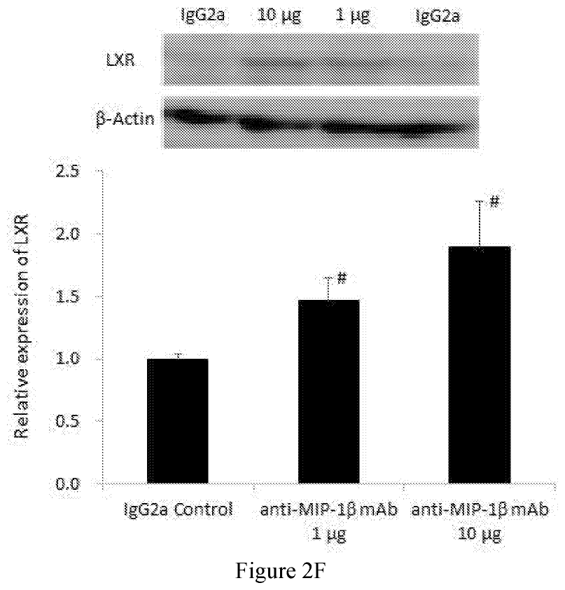

The serum lipids in ApoE KO mice fed a Western diet from 5 weeks of age until 16 weeks of age were examined. After a 4-week 10 μg anti-MIP-1β antibody treatment, there was a significant 9.7% decrease in serum total cholesterol compared to IgG control group (FIG. 2B); The serum triglyceride remains unchanged and was less than IgG control group (˜20%) (FIG. 2C). And non-HDL level was less than IgG control group (˜10%) (FIG. 2D). However, in 1 μg anti-MIP-1β antibody treatment, the effect of lower lipid profile was not significant (FIGS. 2B, 2C and 2D). On the other hands, there was a significant 8.7% decrease in blood glucose after 10 μg anti-MIP-1β antibody treatment compared to IgG control group (FIG. 2A).

MIP-1β inhibition significantly increased LXR expressions in liver tissues in ApoE KO mice (FIG. 2F). The above data showed that MIP-1β inhibition could modify lipid profile via upregulating LXRs and attenuate the elevate trend of blood sugar in atherosclerotic mice.

The metabolic data were provided in Table 1; wherein the data were means±SD (n=6 in each group); TCHO represents total cholesterol; TG represents triglyceride; Non-HDL represents non-high-density lipoprotein (# P<0.05, ## P<0.0/compared with the IgG₂ₐ isotype control group).

TABLE 1
Metabolic data in normal and atherosclerosis mice

| | | | 1 μg MIP-1β | 10 μg MIP-1β |
|---|---|---|---|---|
| | | IgG₂ₐ isotype | antibody treatment | antibody treatment |
| | Normal group | control group | group | group |
| Baseline of the study | | | | |
| (age of 12 weeks) | | | | |
| Body weight (g) | 27.3 ± 1.8 | 29.98 ± 2.86 | 31.97 ± 4.17 | 31.1 ± 3.51 |
| Blood glucose (mg/dl) | 143 ± 16.7 | 154 ± 38.97 | 153.43 ± 23.77 | 153.71 ± 27.72 |
| TCHO (mg/dl) | 76.11 ± 5.46 | 1067.33 ± 332.67 | 1062 ± 126.57 | 1073.33 ± 208.71 |
| TG (mg/dl) | 85.67 ± 9.48 | 110 ± 42.85 | 109.6 ± 30.84 | 110 ± 37.15 |
| Non-HDL (mg/dl) | 2.8 ± 2.51 | 756.8 ± 212.71 | 750.67 ± 61.13 | 757.6 ± 116.99 |
| End of the study | | | | |
| (age of 16 weeks) | | | | |
| Body weight (g) | 27.4 ± 1.6 | 31.05 ± 2.54 | 33.82 ± 5.27 | 32.98 ± 3.72 |
| Blood glucose (mg/dl) | 154.6 ± 12.8 | 239.5 ± 35.67 | 229.57 ± 22.39 | 218.83 ± 23.74^{#} |
| TCHO (mg/dl) | 81 ± 5.75 | 1140.8 ± 258.7 | 1102 ± 261.37 | 1029.33 ± 265.87^{#} |
| TG (mg/dl) | 94.83 ± 3.69 | 147.2 ± 58.02 | 133.29 ± 37.47 | 117.5 ± 25^{#} |
| Non-HDL (mg/dl) | 3.67 ± 2.85 | 912.8 ± 253.15 | 870.4 ± 207.27 | 817.33 ± 252.25^{#} |

### Example 4: Effect of MIP-1β Depletion on Atherosclerotic Plaque Development

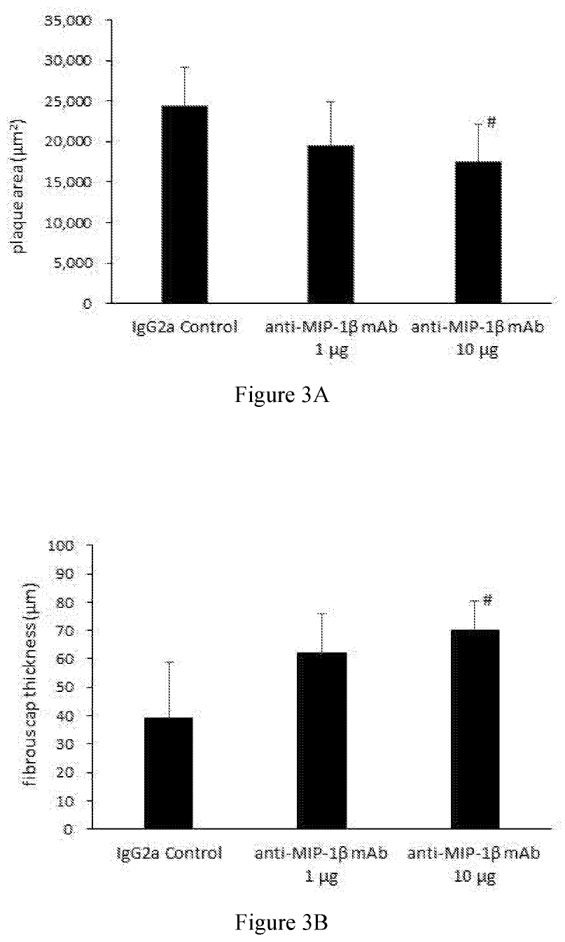

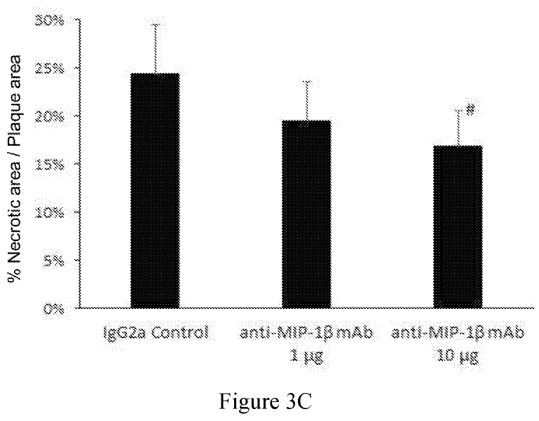

To examine the impact of MIP-1β on atherosclerosis in this model, 12-week-old ApoE KO mice were given anti-MIP-1β for 4 weeks. As shown in FIG. 1A-C, there were elevated levels of MIP-1β in serum and aorta of IgG control group. After 4-week antibody treatment, the value of MIP-1β was reducing. To further understand the effect of the anti-MIP-1β antibody on atherosclerotic plaques. The atherosclerotic lesion area was analyzed and quantified on cross-sectional aortic root staining with HE staining. As compared to that in IgG control group, the atherosclerotic lesion areas were significantly attenuated by 10 μg antibody treatment, for 4 weeks in ApoE KO mice (˜28%) (FIG. 3A).

### Example 5: Effect of MIP-1β Depletion on Atherosclerotic Plaque Quality

Rupture of the fibrous cap is considered to be the critical event that leads to thromboembolic complications in atherosclerotic coronary and carotid artery disease³³. The characteristic feature of ruptured plaques is a thin fibrous cap with a higher ratio of macrophages to vascular smooth muscle cells (VSMCs) covering a large, lipid-rich, collagen-poor necrotic core³⁴. Therefore, this study measured the thickness of the fibrous cap and the size of the lipid-rich necrotic core. Treatment with anti-MIP-1β antibody visibly increased fibrous cap thickness (˜78% increase compared to IgG control) in the aorta (FIG. 3B) and the anti-MIP-1β antibody group exhibited significantly smaller necrotic areas (˜25% decrease compared to IgG control) (FIG. 3C).

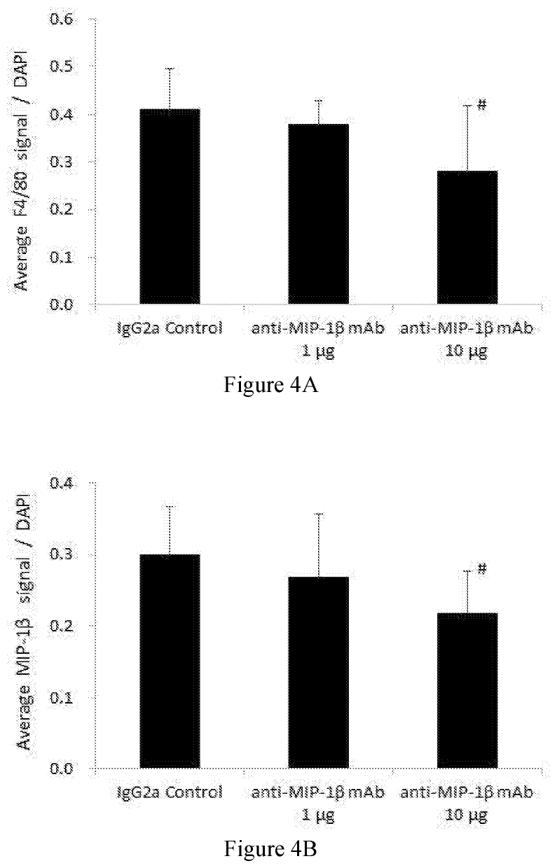

Because increased numbers of inflammatory cells are implicated in plaque vulnerability³⁵, this study next examined macrophage infiltration into plaques. Levels of immunoreactive macrophage marker F4/80 show that macrophage content within plaques was decreased in 10 μg anti-MIP-1β antibody-treated groups (˜40% decrease compared to IgG control) (FIG. 4A). The MIP-1β level was also reduced in plaques (˜21% decrease compared to IgG control) (FIG. 4B).

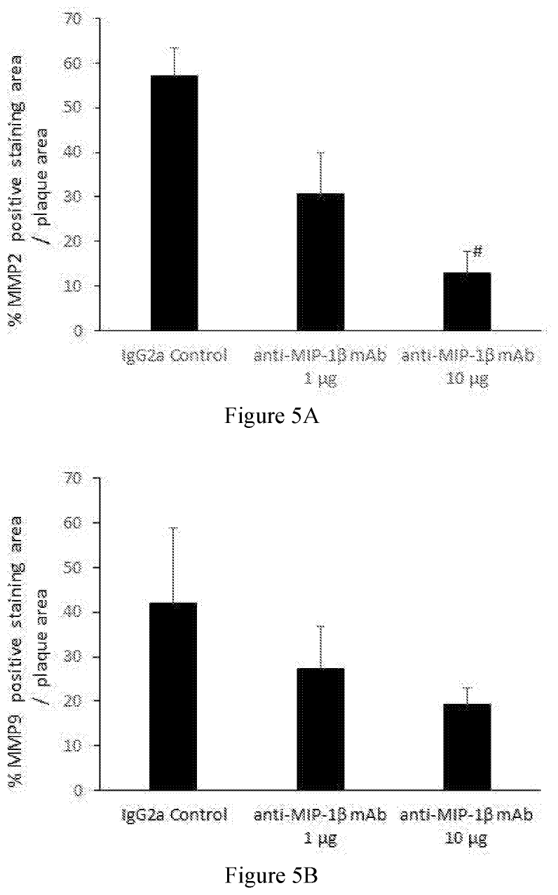

In plaque vulnerability, MMPs are importance because they directly degrade ECM components and are efficient at neutral pH³⁴. Among them, MMP-2 actively degrade intact fibrillar collagens and have a special role in weakening plaques. Destruction of elastin, especially by MMP-9, appears to have a role in outward remodeling and aneurysm formation³⁶. Therefore, this study examined MMP-2 and MMP-9 expressions within plaques and found that both of them were decreased in 10 μg anti-MIP-1β antibody-treated groups (MMP-2: ˜77.2%; MMP-9: ˜54% decrease compared to IgG control) (FIGS. 5A and 5B).

The descriptions and claims as provided should be understood as of demonstrative purpose instead of limitative in any way to the scope of the present invention.

1. A method for preventing, arresting, reversing or treating atherosclerosis, comprising a step of: administering to a subject in need thereof a therapeutically effective amount of a macrophage inflammatory protein-1 beta (MIP-1β) inhibitor.

2. A method for preventing or treating an inflammatory cardiovascular disease or disorder, comprising a step of administering to a subject in need thereof a therapeutically effective amount of a MIP-1β inhibitor.

3. The method of claim 2, wherein the inflammatory cardiovascular disease or disorder is selected from the group consisting of hyperlipidaemia, hypercholesterolaemia, heart attack, stroke, and coronary heart disease.

4. The method of claim 1, wherein the therapeutically effective amount of the anti-MIP-1β inhibitor is the amount sufficient to reduce atherosclerotic lesions or plaques.

5. The method of claim 1, wherein the therapeutically effective amount of the anti-MIP-1β inhibitor is the amount sufficient to retard the progression and promote the stabilization of atheroma plaques.

6. The method of claim 1, wherein the therapeutically effective amount of the anti-MIP-1β inhibitor is the amount sufficient to lower blood lipids, triglyceride, cholesterol and non-high-density lipoprotein.

7. A method for lowering blood lipids, triglyceride, cholesterol or non-high-density lipoprotein, comprising a step of administering to a subject in need thereof a therapeutically effective amount of a MIP-1β inhibitor.

8. A method for treating or preventing atherosclerosis or, the method comprising the steps of:
(1) providing a sample of the subject and determining the level of MIP-1β in the sample, and
(2) administering to said subject, if the subject is found to have a higher level of MIP-1β in the sample than a normal level of a healthy population, an therapeutically effective amount of a MIP-1β inhibitor.

9. The method of claim 1, wherein the MIP-1β inhibitor is an anti-MIP-1β antibody, or a fragment thereof.

10. The method of claim 8, wherein the fragment is a Fab, F(ab′) or F(ab′)₂, or single-chain variable fragment (scFv).

11. The method of claim 1, wherein the MIP-1β inhibitor is a binding protein or peptide which is capable of binding to MIP-1β, or a fragment thereof.

12. The method of claim 10, wherein the binding protein or peptide or a fragment thereof is one capable of binding to an amino acid sequence of SFVMDYYET (SEQ ID NO: 1).

13. The method of claim 10, wherein the binding protein or peptide or a fragment thereof is one capable of binding to an amino acid sequence of AVVFLTKRGRQIC (SEQ ID NO: 2).

14. The method of claim 8, further comprising administering said subject a second therapeutically active agent.

15. A method for lowering blood lipids, triglyceride, cholesterol or non-high-density lipoprotein, comprising a step of administering to a subject in need thereof a therapeutically effective amount of a MIP-1β inhibitor.

16. The method of claim 2, wherein the MIP-1β inhibitor is an anti-MIP-1β antibody, or a fragment thereof.

17. The method of claim 7, wherein the MIP-1β inhibitor is an anti-MIP-1β antibody, or a fragment thereof.

18. The method of claim 8, wherein the MIP-1β inhibitor is an anti-MIP-1β antibody, or a fragment thereof.

19. The method of claim 2, wherein the MIP-1β inhibitor is a binding protein or peptide which is capable of binding to MIP-1β, or a fragment thereof.

20. The method of claim 7, wherein the MIP-1β inhibitor is a binding protein or peptide which is capable of binding to MIP-1β, or a fragment thereof.
